(19) United States
(12) Patent Application Publication
(10) Pub. No.: US 2012/0245457 A1
(43) Pub. Date: Sep. 27, 2012

(54) Ultrasound imaging catheters and guidewires with non-interfering and coordinated position and orientation sensors

(76) Inventor: Robert J. CROWLEY, Sudbury, MA (US)

(21) Appl. No.: 13/506,054

(22) Filed: Mar. 24, 2012

Related U.S. Application Data
(60) Provisional application No. 61/465,980, filed on Mar. 25, 2011.

(51) Int. Cl. A61B 8/12; A61M 25/09

(52) U.S. Cl. 600/424

(57) ABSTRACT

An ultrasonic imaging catheter and guidewire system for acoustic imaging and electromagnetic positioning, with low noise, is described. An ultrasonic imaging guidewire uses two opposed imaging transducers laterally disposed near the distal end of an interventional guidewire. The tortuous position of the guidewire in the vasculature is monitored by a proximally positioned perpendicular position and orientation coil. Accurate vascular imaging is afforded by the use of opposed transducers operating alternately, thereby providing an accurate total distance measurement on each side of the guidewire regardless of the position of the guidewire in the lumen.

This relates to inventions and improvements to acoustic imaging catheters and devices employing a movable ultrasound transducer used in combination with electromagnetic field positioning and is based upon Provisional Application No. 61/465,980 filed on Mar. 25, 2011 and is incorporated herein by reference in its entirety.

### BACKGROUND OF THE INVENTION

### Field of the Invention

It has long been recognized that acoustic imaging by use of internal probes has potential use in visualizing conditions of a body, and that electromagnetic fields may be used to locate, orient or display the position of various catheter devices within the body.

For example, Brown et al., U.S. Pat. No. 2,949,910, describes a catheter within a heart which detects heart size by a fixed transducer which emits sound waves and detects the echo return; Born, U.S. Pat. No. 3,938,502, describes an ultrasonic device to be placed within a heart, comprising a catheter of 3 mm outer diameter having a plurality of fixed transducers. Peronneau, U.S. Pat. No. 3,542,014, describes a catheter having a piezoelectric transducer for measuring the size of a blood vessel; Frazer, U.S. Pat. No. 4,176,662, describes an endoscope employing a transmitter of ultrasonic energy waves so that its position within a body can be determined; Green et al., U.S. Pat. No. 4,327,738, describe an endoscope, having a transducer which is inserted into a body cavity such the stomach; Trimmer et al., U.S. Pat. No. 4,431,006, and Leeny et al., UKA 2,175,828, describe solid needles, for detecting sound waves, which may be inserted within a body; Silverstein et al., U.S. Pat. No. 4,462,408, describe an endoscope having an array of ultrasonic transducers which can be inserted into a body cavity such, as the stomach; and Pourcelot et al., U.S. Pat. No. 4,605,009, describe an endoscopic probe suitable for studying coronary arteries. Other internal probes have been proposed which cause movement of the ultrasonic beam to produce images of body features, of which the following are examples.

Eggleton et al., U.S. Pat. No. 3,779,234, describes an ultrasonic catheter for placement through an esophagus or chest wall. The catheter rotates at 360 rpm and has four transducers each having a diameter of 5 mm. The transducers are focused horizontally with an effective depth of penetration of about 15 cm. The housing of the catheter rotates and “may be a flexible stainless steel shaft constructed of right and left helices to maximize the torsional stiffness of the rotating assembly with the stationary housing. [such as produced by S. S. White and Company]”.

Baba, U.S. Pat. No. 4,374,525, describes an ultrasonic device which may be placed within a body cavity and against a body wall. The transducers are rotated at between 500-900 rpm. The inserted section is generally rigid, but flexible enough to be placed against a body wall.

Suwaki et al., U.S. Pat. No. 4,375,818, describe an endoscope having an ultrasonic transducer suitable for positioning within a celiac cavity. The transducer can be rocked by a drive motor adjacent to it.

Ando et al., U.S. Pat. No. 4,391,282, describe an ultrasonic apparatus for celiac cavity insertion, for examination of the heart and pancreas. “[A] flexible power transmission member such as a coil wire or the like . . . ” is provided.

Nakada et al., U.S. Pat. No. 4,558,706, describe an ultrasonic and optical device. The ultrasonic pulse is rotated by a mirror held on a “soft shaft formed of two inside and outside wound layer coils. These inside and outside coils are wound reversely to each other so that the outside diameter will not vary with the rotating direction and the rotation will be able to be effectively transmitted when curved.” Gas bubbles generated within the catheter are removed through a conduit passing from the housing chamber to an outside chamber.

Kondo et al., U.S. Pat. No. 4,572,201, describe an intraluminal ultrasonic scanner which can be inserted into the stomach or pancreas The transducer is secured to a rotary shaft connected to a flexible shaft.

Andou et al., U.S. Pat. No. 4,674,515, describe an ultrasonic endoscope suitable for placement in a body cavity.

DE 3,619,195, appears to describe an ultrasonic catheter with a coil-spring driven magnet, which, by magnetic coupling, in turn causes rotation of a transducer.

Intravascular ultrasound using a high frequency transducer in a catheter placed in an artery was first described by Penelope J Handley. Handley describes a rotating transducer and mirror combination capable of producing a series of cross sectional images that can be assembled into a pseudo 3D image representation. The design was accomplished before adequate imaging electronics were available for rapid image formation.

Martinelli describes an electromagnetically tracked, manually scanned transducer system that uses an ultrasound transducer adjacent to an electromagnetic sensor. The electromagnetic sensor is a coil that picks up an electromagnetic field generated outside a patient using a plurality of coils. The catheter probe is moved manually through the lumen, building up a stored acoustic image.

Crowley, et al, U.S. Pat. No. 4,951,677 describes an intravascular ultrasound system employing a spinning driveshaft and single coaxial cable with a distally mounted transducer. The transducer directly scans a lumen wall and is carried inside a slidable acoustically transparent sheath. This is the most common form of intravascular ultrasound in use today and provides low noise imaging due to its coaxial, symmetrical mechanical and electrical properties.

Hamm et al describe a guidewire sized transducer and housing that simulates the action of a guidewire and an ultrasound catheter. The invention is complex and expensive.

Strommer el all describes electromagnetic sensing and positioning of interventional catheters in the body using external fields picked up by those catheters and signal lines routed through catheters to a connection to a dedicated medical positioning system computer and display system.

To date, combined catheter based intravascular ultrasound, or IVUS, with electromagnetic (EM) sensor systems has been problematic. Noise artifacts occur when harmonics and products of the EM field enter signal lines of the ultrasound signal. Low frequency EM signals that are generally in the 5 to 100 KHz range mix and modulate with pulse components of the ultrasonic transmitter, producing large amounts of unwanted noise in the receive passband, which is generally in the 5-100 MHz range. The problem is compounded as the device is made smaller in the scale of guidewires.

Larger than desired catheters and guidewires result from adding more signal lines to accommodate both ultrasonic and EM sensors. Each signal line comprised of a miniature coaxial cable adds bulk and cost to the assembly, sometimes referred to as a probe, which is used with an external delivery sheath, both which are typically used together as a single use disposable device. Various manipulable transducer equipped guidewires and catheters having measurement, flow, doppler and imaging capability have been described or attempted, but none have the low profile, simplicity, and practicality required for successful guidewire based imaging.

Wider effective use of acoustic imaging catheters and guidewires would occur, especially in the vascular system and more particularly, the coronary arteries, if such a system could be considerably smaller, have good image fidelity, low noise, and be simple, inexpensive and dependable, and provide more meaningful image and spatial information.

### BRIEF SUMMARY AND OBJECTS OF THE INVENTION

It is the object of the invention to produce a practical, inexpensive, simple and effective device and method for inspecting and analyzing an artery or duct in the human body. It is one object to provide this function in a guidewire form that can be used with other interventional therapeutic devices, such as balloons, stents and atherectomy cutters. It is another object of the invention to overcome certain topological errors that occur when an interventional device is placed in a curved artery. It is a further object of the invention to produce a device having a slim profile, easy manufacture, and relatively low parts count, which add cost.

In one embodiment, an interventional guidewire having a proximal and distal end is equipped with a pair of opposed, separately connected ultrasound transducers radiating perpendicular to the long axis of the guidewire near the distal end, and at least a pair of axially separated low profile ultrasonic pickup coils are disposed within the profile of the guidewire near the proximal end, the guidewire having no enlargement throughout its length.

In one embodiment, the guidewire as previously described above is equipped with an electromagnetic pickup coil near the opposed transducers, and a third embedded pickup coil near the proximal end and spaced axially from the ultrasonic pickup coils.

In one aspect, the method of imaging an artery comprises: Providing a guidewire having a plurality of acoustic transducers, at least two of the transducers pointing in generally opposite directions and operated in pulse-echo mode, advancing the guidewire through a region of interest in the body, monitoring at least one degree of freedom of the guidewire position, and accumulating data for subsequent display of the data as an ultrasound image.

In certain preferred embodiments, the transducer comprises a single transducer element directed at an angle to the axis of the drive shaft, and an electromagnetic sensor, such as a wound coil of fine wire, the two being connected in either parallel or series configuration, yet isolated by electrical impedance and frequency of operation, as well as time of operation through system control, to minimize interference between ultrasound and EM detection.

In certain further improvements to the current art, the probe carrying the transducer and the EM sensor has a distal portion, a central signal wire such as a shielded coaxial cable, and a proximal, rotatable connector that is indexable to a particular position relative to a driving motor. Such an indexable system is equivalant to that produced by Diasonics corporation, Hewlett Packard Corporation, and Boston Scientific Corporation in 1988, and commercialized in the form of Sonicath™ catheters for use in arteries. In the improvements, the signal line, comprising a single coaxial cable, is caused to carry both high frequency IVUS signals and low frequency P&O information back to the imaging electronics without interference between those signals, thereby preserving image quality and not adding to the diameter of the finished device, which is important to safe, convenient and economic delivery to the point of interest, such as a coronary artery.

Various features and advantages of the invention will become apparent through the description, drawings and claims. Drawings are not necessarily shown to scale, emphasis being placed on illustrating the features of the invention. In various drawings, like numbers are mostly used when referring to the same feature, component, part or or group, for the sake of clarity and consistency.

The invention thus comprises a body insertable imaging device for the production of data and subsequent images therefrom consisting of an elongate, hand manipulable device carrying at least two ultrasound transducers and an electromagnetic position function operably oriented with respect to at least one transducer near a distal portion of the imaging device, and a proximally located three channel connector attached in electrical communication to an imaging display. The imaging device preferably utilizes time division to reduce the likelihood of signal interference. The imaging device preferably utilizes time-division operation controlled by the imaging display. The imaging device is effective to provide a cross sectional view gathered from the advancement of the imaging device through an artery. The cross sectional view may include a longitudinal view. The electromagnetic position function is incorporated into at least one of the ultrasound transducer assemblies.

The invention also comprises a guidewire for the production of ultrasonic images from within a portion of a living being and a position sensor located near a distal tip of the guidewire having a pair of separate, opposing acoustic transducers aimed in opposite directions. The time-division data collection is utilized to reduce the likelihood of signal interference. The guidewire may preferably further consist of a low profile proximal magnetic connection system having primary and secondary windings.

The invention also comprises a method of imaging an artery comprising one or more of the following steps: providing a guidewire having a plurality of acoustic transducers, at least two of the transducers pointing in generally opposite directions and operated in pulse-echo mode, advancing the guidewire through a region of interest in the body, monitoring at least one degree of freedom of the guidewire position, and accumulating data for subsequent display of the data as an ultrasound image. The at least one degree of freedom of the guidewire position is detected by an electromagnetic device incorporated in the guidewire and an external electromagnetic position system operating in time division mode.

### BRIEF DESCRIPTION OF THE DRAWINGS

FIG. 1a is a top view of the distal end of an imaging guidewire showing opposed, offset acoustic transducers and an axial view of a coil.

FIG. 1b is a side view of the distal end of an imaging guidewire showing a cross sectional view of the coil.

FIG. 2a is a side view of the proximal end of an imaging guidewire showing both ultrasonic signal and low frequency position signal carrying coils mounted flush with the surface.

FIG. 2b is a side view of an imaging guidewire showing distal and proximal ends.

FIG. 3 is a diagram of an artery with a guidewire traversed therethrough, and external electromagnetic field producing coils placed a distance outside of the artery.

FIG. 4 is a screen view of a resultant image of the artery when obtained using the principles of the inventions herein.

FIG. 5 is a diagrammatic view of a guidewire with five degrees of position information indicated on the diagram with arrows.

FIG. 6 is a side view of the proximal end of an imaging guidewire and a coupler for an imaging guidewire.

FIG. 7 is a cross sectional view of a position sensing coil having an end radius of curvature compatible with the outer profile of an imaging wire.

FIG. 8 is a cross sectional, axial view of one acoustic transducer embedded within the profile of an imaging guidewire.

FIG. 9 is a ¾ view of an ultrasound transducer with a position and orientation inductor incorporated within the rear section of the transducer.

FIG. 10a is a schematic diagram of a coaxial fed ultrasound transducer with P&O having a single coaxial cable feedline and a series inductor position sensing coil with a capacitive bypass.

FIG. 10b is a schematic diagram of a coaxial fed ultrasound transducer with P&O having a single coaxial cable feedline and an inductor position sensing coil in parallel with the piezoelectric transducer.

FIG. 10c is schematic diagram of the magnetic commutation scheme for series-connected primary transformers.

FIG. 10d is a schematic diagram of the magnetic commutation scheme for parallel-connected primary transformers.

FIG. 11 is a timing diagram showing the relationship of the ultrasonic signal and the electromagnetic position signal operation.

FIG. 12 is a side view of a rotatable imaging core with a rotary driveshaft, an axial position sensor, and a coaxial cable.

FIG. 13 is a diagram of a rotary transformer commutator and signal paths for both ultrasonic and acoustic RF signals.

### DETAILED DESCRIPTIONS OF THE INVENTION

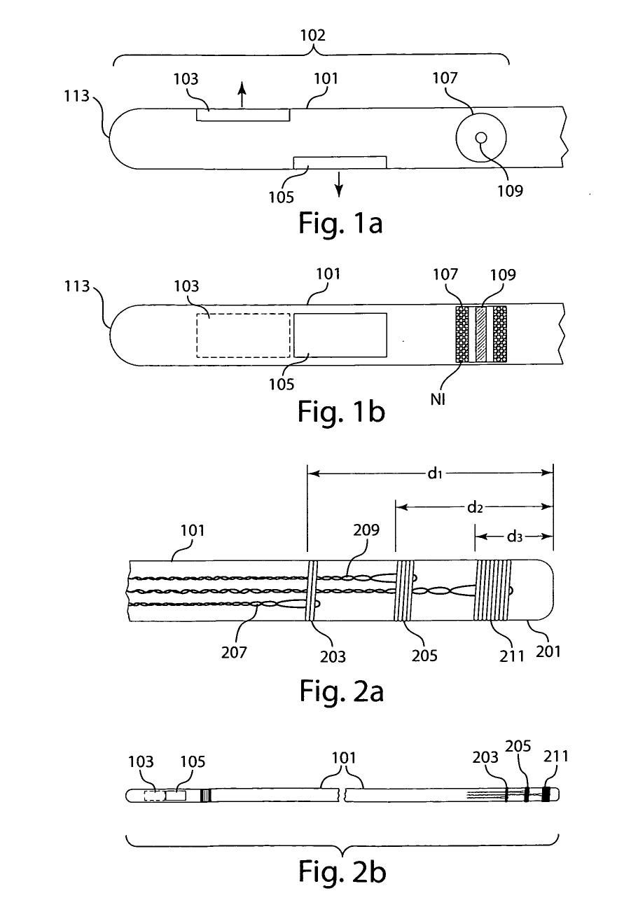

Referring now to FIG. 1a, the top view of the distal end of an imaging guidewire showing opposed, offset acoustic transducers and an axial view of a coil, guidewire body 101 houses two offset acoustic transducers 103 and 105 in an area referred to as tip section 102. The transducers are relatively flat, plate like or panel like in shape, and have an operating frequency in pulse-echo operation of approximately 10 MHz to 100 MHz, the particular center frequency selected by the designer for the optimal depth of penetration vs the frequency dependent attenuation known in intravascular imaging practice and as taught by Crowley et al. The transducers 103 and 105 may be of a layered piezolelectric materials such as lead zirconate titanate, or be of an acoustic film, such as acoustic nanofilm, as taught by Crowley. The requirements are 1. good impulse response, 2. short ringdown, 3. good directivity 4, high sensitivity and 5 wide fractional bandwidth, in the order of 30% or more. Signal wires (not shown) extend from beneath the transducers through the body of the guidewire and around each side of coil 107 that is also embedded in guidewire body 101.

Now more clearly referring to FIG. 1b the side view of the distal end of an imaging guidewire, coil 107 comprises an iron core wound with turns of wire 111. The wire must be fine, and small, and have good conductivity. Such a wire often comprises copper plated with silver, in the order of a filamentary wire having diameters less than 0.0004″ at times, and may be as large as 0.001″. The winding of such fine wires on coils or bobbins is known in the art to produce inductors useful to the detection of various electromagnetic fields, including orienting electromagnetic fields.

Still referring to FIG. 1b, acoustic transducers 103 and 105 are seen to have a relatively offset position spaced a distance axially, and have a generally rectangular shape, although circles, discs or other shapes may provide acoustic radiation and reception according to the principles of the scientist Chladni. Distal tip 113, here shortened for clarity, may comprise a polymeric, floppy, laterally flexible material such as EVA, or polyethylene, and be fitted with various radioopaque markers (not shown) to help show the guidewire under fluoroscopic observation. Such markers however are generally not necessary due to the radioopaque nature of many backing and substrate materials, such as tungsten, that are used in the construction of ultrasound transducers. The guidewire body 101 may be comprised of a polymeric tube of polyimide, or of a hollow tube of metal, such as nitinol. Distal tip 113 may be comprised of a coil of metal (not shown) as known in the conventional guidewire art.

Now referring to FIG. 2a, a side view of the proximal end of an imaging guidewire showing both ultrasonic signal and low frequency position signal carrying coils mounted flush with the surface, guidewire body is shown in transparent view for clarity. Stub end 201 is finished like any proximal guidewire end, that is, with no bump or larger diameter section that would make it difficult to slide another interventional device, such as a balloon or stent delivery device, over the wire once it is in place in an artery. The use of a polymeric material for body 101 is advantageous to embed a multiplicity of coils. Ultrasonic signal coils 203 and 205 may be wound around a plastic body and embedded with a heat and reflow operation that sinks the wires into the body flush. Only a few turns are needed for adequate coupling of coils 203 and 205, due to the relatively high frequencies, in the order of tens of MHz, involved. Signal wires 207 and 209 extend distally through the guidewire body 101 to the ultrasound transducers located at the distal end. Also on the proximal end of the guidewire body as shown in FIG. 2a is the electromagnetic pickup signal coil 211, which is generally designed to transfer signals in the KHz range, and therefore needs more turns of wire. Coils 203, 205, and 211 may be spaced axially at any convenient fixed and controlled distance(s) D1, D2 or D3, from the stub end 201 to provide a known location for further attachment to other devices (not shown or described in FIG. 2a). Typical axial distances on the order of 0.5 to 1.0″ may be used for the various coils.

Now referring to FIG. 2b, guidewire body 101 is shown straight and may have a length of about 40 inches or more, and a diameter of about 0.014″, or 0.018″, or 0.025″, the diameters selected for optimal use in various locations of the body and for appropriate mechanical and axial stiffness properties. Generally, a larger diameter provides a stiffer body. The use of 0.014″ diameter has become “standard” for use in coronary artery procedures, which are one use of the invention that is particularly important. Still referring to FIG. 2b, the coils 203, 205 and 211, and the transducers 103 and 105, and coil 107, can be seen to have an axially fixed relationship to each other. Though the axial position of each element in a flexible interventional device may be maintained during use, lateral, and radial positions change with the bending of that interventional device. It is important for the operation of the invention to know, in relative terms where the relative positions of the elements are in the intrabody space.

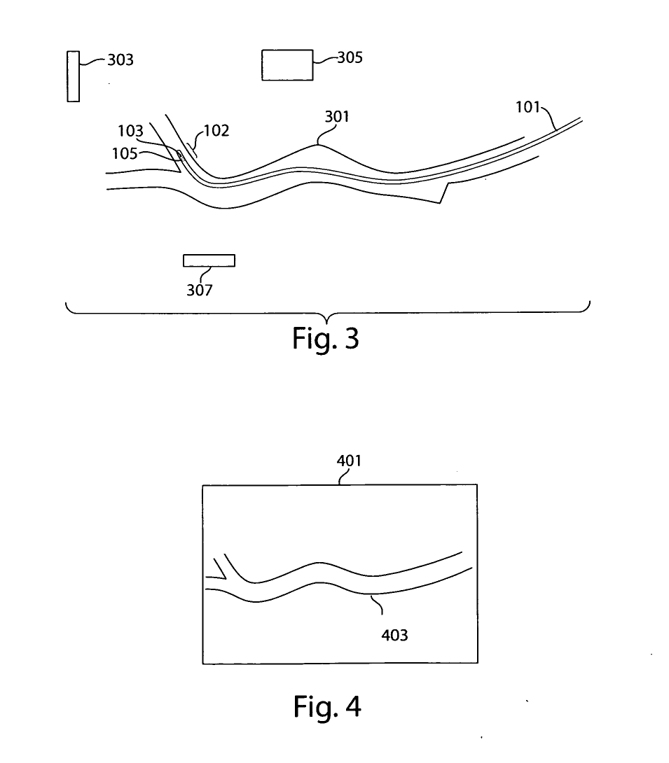

Such a space is shown now in FIG. 3, a diagram of an artery with a guidewire traversed therethrough, and external electromagnetic field producing coils placed a distance outside of the artery. This diagram is purely schematic and does not convey the tortuosity encountered in actual operation,nor the depth, which is not easy to predict. Guidewire body 101 is represented by a line as-drawn. Tortuous artery segment 301 is shown to have various positions and diameters which vary along its length. At any given point along the path, guidewire tip section 102 will achieve a different angular position, as well, as a different coordinate position, and angle relative to its long axis. These positions are commonly referred to as absolute position, and pitch, yaw, and roll. Often these positions and angles are also referred to as “six degrees of freedom”. It is an object of this invention to know at appropriate times at least some of these positions and angles relative to the ultrasound beams, so that more meaningful representative ultrasonic cross sectional images can be calculated and displayed. Alternating current positioning coils 303, 305, and 307 may be positioned relative to X, Y and Z axes outside the body and energized with multiple alternating current signals, which provide identifiable signals of varying phase and intensity at coil 107 that remains fixed relative to tip section 102. Such a system is commercially available from Ascension Technologies, VT, and other Position and Orientation (P&O) system companies that use P&O information for gaming, film making, medical imaging, and military guidance applications. In particular, systems that employ two or more transmitting coils of different orientations and operating frequencies in the 10 to 500 KHz range are most suitable for the operation of this invention.

Still referring to FIG. 3, guidewire tip section 102 with opposing transducers 103 and 105 is shown in artery segment 301 as relatively centered within the lumen of segment 301. Such is usually not the case, the tendency of interventional wires and catheters to move to one side of the lumen. Accurate total cross sectional measurement along a plane defined by the opposed transducers is maintained regardless of the position of the tip section 102, because the total transit distance, wall to wall, of the two opposing beams always add up to the total wall to wall distance, within that plane. In other words, the sum of the two beam distances reflected from the wall of a lumen will always be at or near the total wall to wall distance, regardless if the tip section 102 is toward the left, toward the right, or somewhere in-between. Operation involves simply advancing the guidewire once into the artery. This allows the user to get accurate cross sectional local acoustic information and may be used as an “ultrasound only” wire that provides a cross section image without curves, displayed in a straight line similar to that produced by “Length Mode” or “L” mode IVUS systems that only show straight displays of multiple rotationally scanned live cross section views. Such IVUS systems require the use of a rotating drive cable, which is not needed or desired in an imaging guidewire, but have the advantage of producing a live image in real time of a certain cross sectional area.

Still referring to FIG. 3, this information is then analyzed and plotted according to the detected tip section 102 position located by the use of external alternating current positioning coils 303, 305, and 307. Now referring to FIG. 4, a screen view of a resultant image of the artery when obtained using the principles of the inventions herein, the obtained “L” mode image 401 also shows the curvature, represented in 2D of the displayed artery segment 403. This image is obtained without internal moving parts and with simple opposed transducers and a P&O capability.

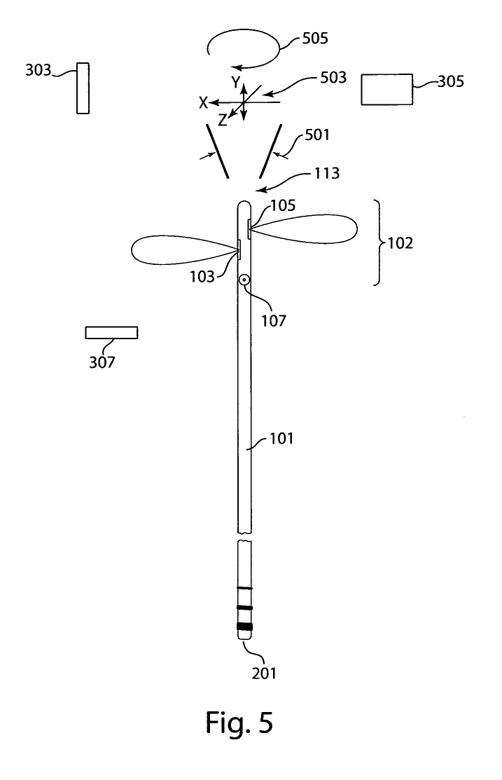

Further understanding of the P&O relationship of the guidewire to the displayed image is shown in FIG. 5, a diagrammatic view of a guidewire with five degrees of position information indicated on the diagram with arrows. Once again showing distal tip section 102, transducers 103 and 105, and coil 107, it can be appreciated that even with some flexibility the relationship, distance and position of these elements remain relatively fixed to each other. As guidewire body 101 is bent, however, various degrees of freedom are obtained, for instance, lateral position 501 may change. Also, as the guidewire is advanced through the body, its position in space X, Y and Z may change. Finally, as the guidewire is advanced, it may be twisted, either intentionally, or unintentionally, about circular axis position 505. In all cases, acoustic transducers 103 and 105 produce opposing beams and an accurate total transit distance indication. In any position, external alternating field coils 303, 305 and 307 are available to transmit signals to coil 107. For the purpose of this invention, coils 303, 305 and 307 shall be deemed to have different frequencies emanating therefrom to be picked up by coil 107, however, it should be understood that different phase signals may be used to discriminate between each coil and its position with respect to the guidewire distal tip section 102.

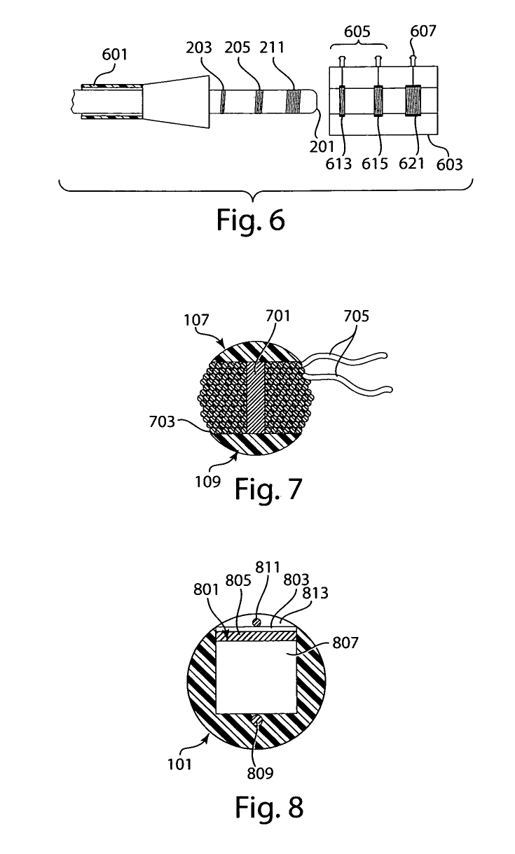

Now referring to FIG. 6, a side view of the proximal end of an imaging guidewire and a coupler for an imaging guidewire, proximal end 201 may be inserted into electrical coupler 603 after interventional device 601, which may be an introducer, a guiding catheter, a balloon catheter, a stent catheter. Coils 203, 205 and 211 line up with pickup coils 613, 615 and 621, respectively, in a close magnetic relationship effective to transfer signal energy in the radiofrequency ranges of KHz and MHz, and are carried out to port pair 605, which correspond to ultrasound signals, and 607, which corresponds to P&O signals. Coupler 603 is slidably and readily attached and detached and may be sealed against water, blood or saline using a polymer coating, such as paint. This arrangement provides and effective and fast connection with no bump on the guidewire that might interfere with other interventional devices, yet conveys P&O and ultrasound signals effectively.

Now referring to FIG. 7, a cross sectional view of a position sensing coil having an end radius of curvature compatible with the outer profile of an imaging wire, certain details of that component can be appreciated. Coil 107 and bobbin 109 have a generally cylindrical iron core 701 and ends 703 with radii that conform to the outline of the interventional device. Fine turns of wire 111 are wound around the core 701 and signal leads 705 are prepared and dressed for installation and routing.

Referring now to FIG. 8, a cross sectional, axial view of one acoustic transducer embedded within the profile of an imaging guidewire, the flat face of the transducer sandwich 801 is recessed slightly below the profile of body 101, since it is necessary to have a generally flat radiating surface for good acoustic beam formation. Lens 803 is of a quarter wavelength thickness and made of a conductive epoxy, such as a silver epoxy, and is bonded to piezo layer 805, which is the active layer than mechanically deforms and produces an electrical signal in response to incoming acoustic vibrations, and also produces an outgoing acoustic pulse when excited with an electrical signal. To make such a transducer, first grind the piezo material to a thickness that resonates at the required frequency, such as 40 MHz. Then, apply an acoustic impedance matching layer, or lens 803, of a material that is near the geometric mean of the acoustic impedance of the piezo material 805, and water. A typical layer thickness is 0.00060″. Then, apply under high pressure and heat, a composite backing layer, 807. Backing layer 807 is also comprised of a conductive epoxy loaded with a dense, sound absorbing material, such as tungsten, or packed multiwalled carbon nanotubes which are dense, conductive, and acoustically lossy. Multiwalled carbon nanotubes such as those described by Crowley in U.S. Pat. Nos. 6,038,060 and 7,099,071, incorporated herein by reference, may be employed. These appear, in bulk, as a black sooty substance, and may be mixed with an epoxy or other binder, or compressed loose and used as a pressure sensor, or to detect a motion, or as a lightwave sensor.

Still referring to FIG. 8, first signal wire 809, shown in cross sectioned axial view, and second signal wire 811, provide connection to the transducer, and may have leads (not otherwise shown in this view) that may be prepared and dressed for installation and routing, or may be attached after the transducer is glued or insert molded into the body and thus forms a stressed portion of the cross section which, due to its inherent tensile strength, does not compromise the integrity of the body. Such insert molding into a body was accomplished by Crowley et al in 1988 at Boston Scientific Corporation, and may be usefully employed here. If there is a desire to fill the depression area 813, it may be filled with low density polyethylene, or polyethylene oxide, for a totally smooth surface contour around the body 101, without seriously degrading or attenuating the acoustic propagation from transducer sandwich 801.

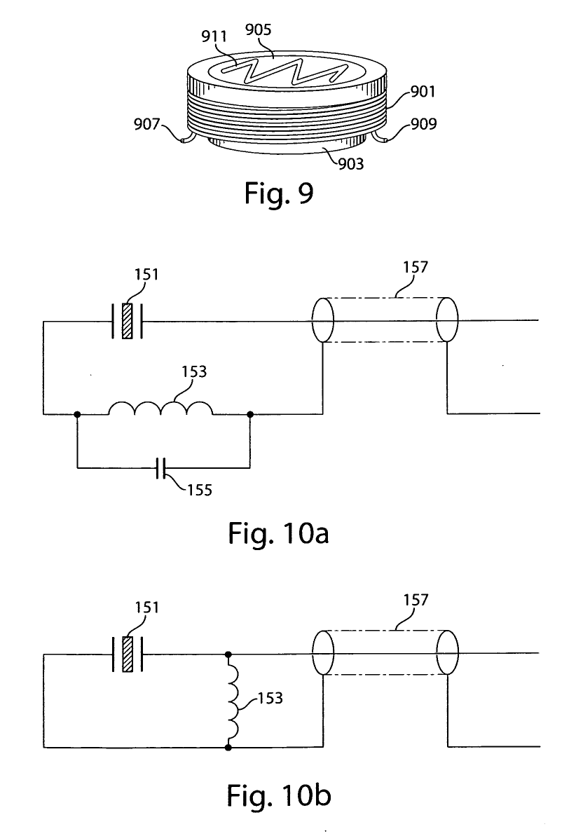

An alternative transducer construction with certain advantages is depicted in FIG. 9, a ¾ view of an ultrasound transducer with a position and orientation inductor incorporated within the rear section of the transducer. Instead of the sandwich construction and use of piezo materials such as lead-zirconate-tinanate, an electrodynamic transducer may be usefully constructed. Coils 901 surround a ferrous stem 903 that has attached to it a tympanic membrane of magnetic, acoustic nanofilm 905. One such acoustic nanofilm construction is described by Crowley in U.S. Pat. Nos. 7,894,619 and 7,900,337, each incorporated herein by reference. This construction, which mimics the operation of many coil driven loudspeakers, is highly advantageous to the invention since both acoustic and electromagnetic (coil) operation is contained in one unit. Ferrous stem 903 may be molded or machined from a ferrite material into the shape of a short, stepped stem with a diameter of about 0.010″ in diameter to about 0.100″ in diameter in a practical manner using ordinary ferrite machining or molding techniques. Coil 901 is wound with very fine copper and or silver wire, having a diameter of less than 0.001″. As few as 8 turns of wire are needed for operation in the high MHz range, such as 40 MHz. However, more turns are desired for optimal pickup of electromagnetic fields as previously described herein. Therefore, a range of turns of coil 901 may be from about 8 turns to about 100 turns. Wire ends 907 and 909 are shown unterminated for clarity, though it should be understood that wire ends 907 and 909 may be dressed and routed through a catheter or guidewire body for conduction of signals therethrough.

Still referring to FIG. 9, operation as an acoustic transducer is as follows: Transmission: Pulse from transmitter electronics causes a rapid magnetic field to be produced in coil 901. Magnetic pulse is aligned in ferrite stem 903 like a solenoid, acting upon tympanic membrane of magnetic nanofilm 905, which rapidly distorts and produces a mechanical pulse that propagates perpendicular to the face of the membrane 905. Because of the low mass and rapid rise time, very high frequencies can be obtained. Reception: Echoes from surrounding tissue arrive at tympanic membrane 905 and produce small vibrations on its surface. Motion of membrane 905 against low voltage energizing the coil 901, which produces a magnetic field, is effective to induce currents along signal lines 907 and 909 as vibrations are received. Operation is similar to a loudspeaker which has both may be used as a loudspeaker and a microphone as known in the ordinary loudspeaker and microphone art, but in a very much smaller space and higher frequency needed for acoustic imaging. Magnetic acoustic nanofilm may be produced by known methods of depositing metals such as nickel, iron, aluminum alloys, and gold in combinations upon strong substrates such as polymers, carbon nanotube supports, or other metals, and have various shapes and corrugations 911 impressed or molded thereon, in order to obtain flexibility, magnetic properties, conductivity and acoustic radiation-reception properties. Such manufacturing is described in detail in U.S. Pat. Nos. 7,894,619 and 7,900,337 by Robert J Crowley and specifically included are embodiments from those patents including the use of high temperature small sized superconducting materials which may be useful to more efficient operation of the inventions described herein, but is not essential.

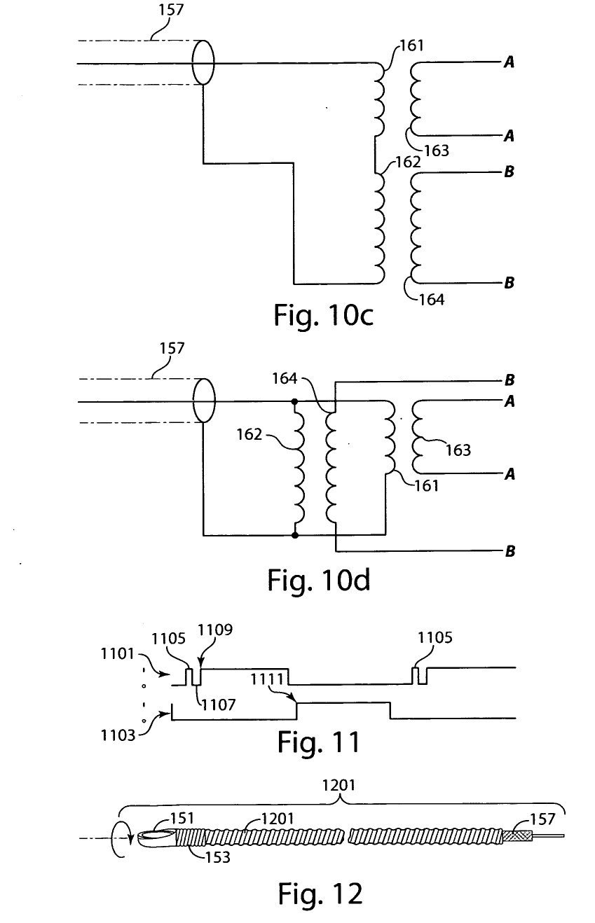

Having thus shown practical constructions to produce and receive acoustic information and receive electromagnetic position information using a coil form structure mounted relative thereto, FIG. 10a-b show ways to conduct the usable signals over a single transmission line, in this case a shielded, coaxial transmission line that is preferred where low noise and resistance to radiofrequency ingress is desired. For the purpose of this explanation, the transducer referred to will be a piezo transducer although it should be understood that other forms of transducers, including electrodynamic types are previously described, may be used according to the same principles.

Referring now to FIG. 10a, a schematic diagram of a coaxial fed ultrasound transducer with P&O having a single coaxial cable feedline and a series inductor position sensing coil with a capacitive bypass, transducer 151 is connected in series arrangement with coil inductor 153. Such an arrangement is useful if the transducer 151 is of a type that can pass low frequency alternating current in the 10-100 KHz range, which may be accomplished using various types of transducers with either high capacitance or low resistivity. Bypass capacitor 155 may be used to tune the circuit so that high frequency RF in the 40 MHz range may pass around coil 153. Elements 151, 153 and 155 are connected as shown to coaxial cable comprising an outer shield and an inner conducting wire.

Series-connected operation may have certain frequency limitations and construction aspects, and parallel operation may be desired.

Referring now to FIG. 10b, a schematic diagram of a coaxial fed ultrasound transducer with P&O having a single coaxial cable and an inductor position sensing coil in parallel with the piezoelectric transducer. Transducer 151 is shown with coil 153 across, in parallel with transducer 151, and leads connected to coaxial cable 157. At high frequencies, the coil 153 is of sufficient inductance to present a high impedance and therefore negligible loss across the signal line to the transducer 151, which operates primarily at frequencies 10× higher than coil 153. Transducer 151 represents a very high impedance and therefore little loss at low frequencies across coil 153. Therefore the two may share a single transmission line 157 by virtue of frequency division.

The connection of single transmission line 157 to further electronics is of importance to aspects of the inventions and described more completely in FIG. 10c-d.

Now referring to FIG. 10c, a schematic diagram of the magnetic commutation scheme for series-connected primary transformers, coaxial cable 157 is shown connected to a series of primary coils 161 and 162. Each primary coil is placed in near proximity to its own secondary coil 163 and 164, respectively, and labeled for further transmission to circuit A-A or to circuit B-B. Together they work as transformers, which are known to have various voltage relationships such as 1:1, or step up, or step down. Generally, larger transformers, with more turns, are needed for low frequencies in the KHz range, and fewer turns in the MHz range.

Series-connected operation may have certain frequency limitations and construction aspects, and parallel operation may be desired.

Referring now to FIG. 10d, a schematic diagram of the magnetic commutation scheme for parallel-connected primary transformers, coaxial cable 157 may be connected, in parallel, to both primary coils 161 and 162. Near proximity secondary coils 163 and 164 still are labeled A-A and B-B for delivery to further circuits, in this case A-A is the high frequency circuit containing mostly ultrasound imaging RF information, and B-B the low frequency circuit containing mostly alternating current P&O information. Having two separate circuits with some isolation between is advantageous for separating, without interference, the two signals thus carried along a single transmission line, and for adapting existing ultrasound-onoy architecture to P&O capability, since it becomes easy to “split off” the Low Frequency AC (LFAC) to a separate, add on electronics.

Frequency division multiplexing of the signals may be effective to separate the two desired signals without undue interference therebetween. As smaller devices are used and more sensitive receivers are desired, this isolation, which may be in the order of 40 dB or more, may be insufficient. Therefore an additional measure is taken to assure non interference between ultrasonic and low frequency AC signals, by use of time division sharing of the single transmission line. Timing diagrams shown herein are not to scale, emphasis being placed on relationship of events.

Referring now to FIG. 11, a timing diagram showing the relationship of the ultrasonic signal and the electromagnetic position signal operation. Up indicates the “on” state. Ultrasonic imaging timing is described in diagram 1101, and P&O timing is described in diagram 1103. Referring now to timing diagram 1101, pulse signal begins and synchronizes, or triggers all events, starting with the production of a short duration, high voltage pulse sent out to the transducer. This duration may be less than 500 nSec. A brief, less than 1 uSec pause 1107 follows, and then receive gate which need only be of a duration to receive acoustic signals from near the transducer, since the artery is rarely larger than 3 mm. The propagation speed of sound in the human body is about 1.5 mm/uSec (1500 meters per second) and must account for two-way propagation. Therefore a duration of 1109 of 4 MSec is would be barely adequate, and perhaps 8 uSec would be generous. Operation of prior art ultrasound catheters use rotary scanning comprising 256 vectors within one sweep, or circle. The transmit pulse to transmit pulse timing is then 1/256 making each vector event 1/256 seconds or 0.0039 seconds, which otherwise can be stated as 3.9 milliseconds—a very long time compared to the short total ultrasonic send receive cycle of about 8 uSec. Even when two opposing transducers are used, such as those described in FIG. 1a-b, there is more than enough time to accommodate both ultrasound images, and the P&O information.

This leaves time for the ultrasound receiver to turn off, and the P&O system to energize and operate.

Once again returning to FIG. 11, P&O timing 1103 turn on occurs at event 1111, well after event duration 1109 is over. 1111 can continue for a long time before it has to turn off for pulse 1105 to resume. Time division multiplexing of image information and P&O information is thus accomplished with this simple timing expedient that may be triggered from an already-present signal found in all ultrasonic imaging systems, including present day IVUS systems. IVUS systems already have angular positioning information by virtue of the driveshaft construction used to produce an imaging core as described in US Patent 677 by Robert J Crowley, incorporated herein by reference.

Such an IVUS transducer used in present day may be fitted with P&O capability, without interference, using the principles thus described.

FIG. 12 is a side view of a rotatable imaging core with a rotary driveshaft, an axial position sensor, and a coaxial cable that shows how this may be accomplished simply. Multifilar driveshaft is comprised of at least two layers of counterwound wires to provide 1:1 torque and has a hollow lumen to accommodate coaxial cable 157 through its length. Coaxial cable 157 may be connected in series or in parallel, as previously taught to both transducer 151 and coil 153, which may be placed axially now that rotary position control is afforded by the multiifilar driveshaft. Coil 153 may be turns of copper wire and may have a diameter corresponding to the diameter of core assembly 1201 for easy slidable insertion into delivery catheters (not shown). The addition of a coil axially spaced in relation to an existing transducer is very economical, and practical, made operational by various applications of the principles of the invention. Special use of the rotary capability of cores is afforded in this instance.

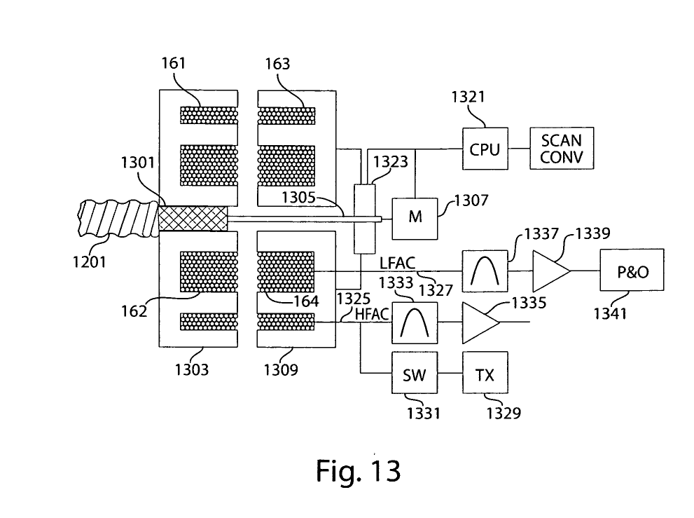

Now referring to FIG. 13, a diagram of a rotary transformer commutator and signal paths for both ultrasonic and acoustic RF signals, certain physical, mechanical and system relationships are described. Multifilar shaft 1201 is terminated in indexed connector 1301 and signal wires routed to the primary windings of coils 161 and 162 as previously described. Coils 161 and 162 are housed in cylindrical ferrite rotary transformer primary cup 1303 which can be rotated along with shaft 1201 by pinion 1305 driven by motor 1307. Air space is provided for free turning of the cup 1303 relative to transformer secondary cup 1309, which remains stationary and houses secondary coils 163 and 164. Controlled by CPU controller 1321, motor 1307 is monitored by angle encoder.

Still referring to FIG. 13, the two signal paths High Frequency AC (HFAC) 1325 and Low Frequency AC (LFAC) 1327 are shown. HFAC 1325 is connected to ordinary ultrasound imaging system electronics consisting of a transmitter 1329, a transmit/receive switch 1331, that produces a pulse as mentioned in the timing diagram, and a bandpass filter 1333 followed by an amplifier 1335. When two transducers are used in sequence, as in the guidewire embodiment of FIG. 1a-b, then two separate transmitters and receivers may be used, or a switch may be used to switch back and forth between the two transducers. The switch has the advantage of knowing which transducer leads and which transducer lags in the event that information becomes important. Time division is sufficient to know which is the “left” and which is the “right” hand transducer in this event, and both may be connected in parallel or series according to the multiplexing principles as taught herein.

LFAC path 1327 is also followed by a bandpass filter 1337 and amplifier 1339, and used with a P&O system 1341 such as described in FIG. 3, and displayed as described in FIG. 4.

What has been described is a substantial improvement to the art of catheter and guidewire imaging, with a practical and well planned signal path, non interfering signals, realistically sized coils and transducers, convenient manual and rotational imaging obtained in an effective manner, and good compatibility with existing P&O and IVUS systems.

I claim:

1. A body insertable imaging device for the production of data and subsequent images therefrom consisting of an elongate, hand manipulable device carrying at least two ultrasound transducers and an electromagnetic position function operably oriented with respect to at least one transducer near a distal portion of said imaging device, and a proximally located three channel connector attached in electrical communication to an imaging display.

2. The body insertable imaging device of claim 1, wherein the imaging device utilizes time division to reduce the likelihood of signal interference.

3. The body insertable imaging device of claim 1, wherein the imaging device utilizes time-division operation controlled by the imaging display.

4. The body insertable imaging device of claim 1, wherein the imaging device is effective to provide a cross sectional view gathered from the advancement of the imaging device through an artery.

5. The body insertable imaging device of claim 3, wherein the cross sectional view includes a longitudinal view.

6. The body insertable imaging device of claim 1 wherein the electromagnetic position function is incorporated into at least one of the ultrasound transducer assemblies.

7. A guidewire for the production of ultrasonic images from within a portion of a living being and a position sensor located near a distal tip of the guidewire having a pair of separate, opposing acoustic transducers aimed in opposite directions.

8. A guidewire for the production of ultrasonic images from within a portion of a living being as recited in claim 7, wherein time-division data collection is utilized to reduce the likelihood of signal interference.

9. A guidewire for the production of ultrasonic images from within a portion of a living being as recited in claim 8, further consisting of a low profile proximal magnetic connection system having primary and secondary windings.

10. The method of imaging an artery comprising: Providing a guidewire having a plurality of acoustic transducers, at least two of the transducers pointing in generally opposite directions and operated in pulse-echo mode, advancing the guidewire through a region of interest in the body, monitoring at least one degree of freedom of the guidewire position, and accumulating data for subsequent display of the data as an ultrasound image.

11. The method of imaging and artery as described in claim 10, wherein said at least one degree of freedom of the guidewire position is detected by an electromagnetic device incorporated in the guidewire and an external electromagnetic position system operating in time division mode.
